(19) United States
(12) Patent Application Publication
(10) Pub. No.: US 2018/0350266 A1
(43) Pub. Date: Dec. 6, 2018

(54) METHOD FOR PRODUCING ANATOMICAL MODELS AND MODELS OBTAINED

(71) Applicant: Dario GARCÍA CALDERON, Altorreal (Molina de Segura) Murcia (ES)

(72) Inventor: Dario GARCÍA CALDERON, Altorreal (Molina de Segura) Murcia (ES)

(21) Appl. No.: 15/774,179

(22) PCT Filed: Nov. 4, 2016
(86) PCT No.: PCT/ES2016/000123

§ 371 Date: May 7, 2018

(30) Foreign Application Priority Data
Nov. 5, 2015 (ES) P201500800

(51) Int. Cl. G09B 23/30; A61F 2/30; A61B 34/10

(52) U.S. Cl. CPC G09B 23/30; A61F 2/30942; G05B 19/4099; A61B 2034/102; A61B 34/10

(57) ABSTRACT

A method for producing anatomical models and models obtained are disclosed. The method includes obtaining information by diagnostic imaging; obtaining a three-dimensional, computerised model of the anatomical structure; and performing the following steps: designing a negative mould; printing the negative mould in 3D; printing in 3D rigid pieces of internal elements of the model provided; placing the pieces in the mould; closing and sealing the mould; injecting a soft material into the mould; and removing same from the mould. The anatomical model is a liver with hepatobiliary vasculature and tumours made from the rigid parts and hepatic parenchyma made from the soft material, or it is a mammary gland with a tumour and muscular tissue made from the rigid parts, mammary tissue made from the soft material and an external covering as skin.

### OBJECT OF THE INVENTION

The invention, as expressed in the title of the present specification, relates to a method for manufacturing anatomical models and to the obtained models, providing features, which will be described in detail below, which involve a novelty in the current state of the art within its field of application.

More particularly, the object of the invention is directed to a method of making anatomical models of human organs, or parts thereof. In particular, soft organs, such as liver or breast models, which have utility in teaching and different disciplines of the medical sector, such as the planning and simulation of surgeries, incorporating significant innovations and advantages over current manufacturing processes thereof. A second aspect of the invention is the same anatomical models obtained from such manufacturing process.

### FIELD OF APPLICATION OF THE INVENTION

The application field of this invention is marked within the field of medicine. The invention also focuses on the field of the industry dedicated to the manufacture of anatomical models intended for teaching or other disciplines of the medical sector, such as the planning and simulation of surgeries.

### BACKGROUND OF THE INVENTION

It is a known fact that anatomical models are physical representations of different human body structures such as organs or limbs. They have application, among other fields, in teaching, being characterized in that they serve to aid in the structural and functional three dimensional understanding of the structures of the human body, and scheduling and/or simulation of surgical procedures that allow the surgeon to be trained in the procedure by solving problems inherent therein.

In the state of the art, the manufacture of standard anatomical models is known, i.e., models that in no case correspond to a particular patient and which are manufactured by using molds.

A method of making specific anatomical models for each patient by a first stage is disclosed in document ES2523419A1, not considered inventive activity, which consists in the generation of a three-dimensional model of the structure of which the anatomical model is to be obtained from a patient's image diagnosis and a second stage where the three-dimensional model is printed directly with a 3D printer.

However, direct printing of the part limits the production of soft organ models, since the non-rigid 3D printing materials have hardness limitations and are expensive. In this way, anatomical models of soft organs, such as liver or breast, would not be possible with the actual consistency of the organ. It is only possible to obtain hard materials without the possibility of emulating surgical cutting and suturing procedures, or with minimum hardness levels of “shore A27”, which is not sufficient to emulate the consistency and deformation of these organs.

The scientific publication Zein, N. N. et al. Three-dimensional print of a liver for preoperative planning in living donor liver transplantation. Liver Transpl. 19, 1304-1310 (2013) prints in 3D a liver using this method. Other scientific publications such as Valverde, I. et al. 3D printed cardiovascular models for surgical planning in complex congenital heart diseases. Journal of Cardiovascular Magnetic Resonance 17, P196 (2015); Tam, M. D. B. S., Laycock, S. D., Brown, J. R. I. & Jakeways, M. 3D printing of an aortic aneurysm to facilitate decision making and device selection for endovascular aneurysm repair in complex neck anatomy. J. Endovasc. Ther. 20, 863-867 (2013); Wang, J.-Q. et al. Printed Three-dimensional Anatomic Templates for Virtual Preoperative Planning Before Reconstruction of Old Pelvic Injuries: Initial Results. Chinese Medical Journal 128, 477 (2015) are about 3D printing of different anatomical models.

The object of the present invention is thus, to develop an improved method of manufacturing anatomical molds in order to solve the drawbacks described above. It should be pointed out that the applicant does not have knowledge of the existence of any other method or invention of similar application that have similar features to those which characterize the invention in that it is claimed.

### EXPLANATION OF THE INVENTION

Thus, the method of making anatomical models of the invention is a novelty within its field of application, since, due to its implementation, the aforementioned objectives are successfully achieved, the details and characterizations thereof being conveniently collected in the final claims included in this disclosure.

In particular, the invention relates, as pointed out above, a method for the manufacture of anatomical models of soft organs, or parts thereof, in particular organ models such as liver or breast, and, preferably, of particular patients, comprising the following essential steps:
Scanning by image diagnostic techniques, such as CAT (Computerized Axial Tomography) with or without vascular reconstruction, NMR (Nuclear Magnetic Resonance), Echography, or similar technique of the anatomical structure of which the anatomical model is to be manufactured.
Using medical specific image processing software that allows selecting (segmenting) the different elements of the anatomical structure of interest from the images obtained in the aforementioned techniques and then obtaining the three-dimensional computerized model of the anatomical structure, which is imported into stereo-lithography (.stl) or the like, valid for 3D printing.
Manufacturing of the anatomical model assembly by means of 3D printing:
   Firstly, the manufacture of the negative mold of the anatomical structure of interest, which is designed with a computer-aided design (CAD) program.
   The manufacture is then made by 3D printing of rigid or semi rigid material pieces that make up certain Internal elements of the anatomical structure, as stated in each case, the need exists for or not to be traversed with medical Instruments, which are placed inside the mold.
   Thereafter, soft materials such as silicones or silicone gels are injected into the negative mold with the hard elements, after considering the consistency of the organ, in order to obtain one or more pieces of such soft material making up the outer part of the anatomical model.
   Finally it is demolded thus obtaining the anatomical model of interest in which two types of elements are clearly distinguished, the internal elements made of rigid or semi rigid pieces, preferably opaque materials and differentiating colors, and external elements made of soft, preferably transparent material, thereby allowing viewing of the internal elements and access thereto with medical instruments.

It is important to note that the method of the invention is particularly concerned with the manufacture of non-prosthetic pieces, and in any case with those which are made of biological material, since the purpose of the obtained model is not intended to be inserted into the interior of the body, since its application, as pointed out above, is in teaching and different test disciplines or study of the medical sector.

Thanks to these characteristics, health and teaching professionals are provided with customized anatomical models for each patient in an economical way and with more functionalities than current anatomical models as they allow emulating surgical procedures.

Other features and advantages of the manufacturing method of this invention will be apparent from the description of a preferred embodiment, but not exclusive, which is illustrated by way of non-limiting example in the attached drawings.

In any event, the disclosed method represents an innovation of heretofore unknown characteristics for the intended purpose, reasons that together with its practical utility, establish the amount of foundation sufficient to obtain the exclusivity privilege that is hereby requested.

### DESCRIPTION OF THE DRAWINGS

In order to complement the description being made and in order to provide a better understanding of the characteristics of the invention, a set of drawings is attached to this specification, as an integral part thereof, represented as follows by way of non-limiting example:

FIG. 1.—It shows a schematic representation of the image of the three-dimensional reconstruction of the liver of a patient, obtained by specific software from diagnostic images, wherein the different parts of the organ are seen, and which constitutes one of the initial phases of the method of the invention, in an example thereof for obtaining a liver model.

FIG. 2.—It shows a schematic perspective view of the negative mold of the anatomical model of the liver shown in the preceding figure, including several of the internal elements thereof constructed of rigid material, such mold being represented only partially and open, in order to show such elements in their phase prior to injection of the soft material.

FIG. 3.—In a view similar to the above, also showing the open mold, in this case as with the soft and dry soft material already applied, in the demolding phase of the final piece.

FIG. 4.—It shows a schematic representation of the anatomical model obtained as a final result of the method of the invention, showing its configuration and the comprising parts and elements.

FIG. 5.—It shows a view of the representation of the three-dimensional reconstruction of the breast of a patient, and which constitutes one of the initial phases of the method of the invention, in a new example thereof for obtaining an anatomical breast model.

FIG. 6.—Another phase of the process is shown where the negative mold is placed between supports and an internal element, in this case a tumor, is held by rods for insertion of the soft material.

And FIG. 7.—It shows a perspective representation of the piece of anatomical breast model obtained with the method of the invention.

### PREFERRED EMBODIMENT OF THE INVENTION

In view of the aforementioned figures, and according to their numbering, a non-limiting exemplary embodiment of the method for the manufacture of anatomical liver models (A) and breast (B) can be seen, which comprises the following:

### Example (A) for Anatomical Models of Liver

The method of making anatomical models of liver comprises, in the first stage, obtaining information about the patient's liver by an image diagnosis such as a CAT (Computerized Axial Tomography) with or without vascular reconstruction, NMR (Nuclear Magnetic Resonance), Echography, Cholangiography or the like.

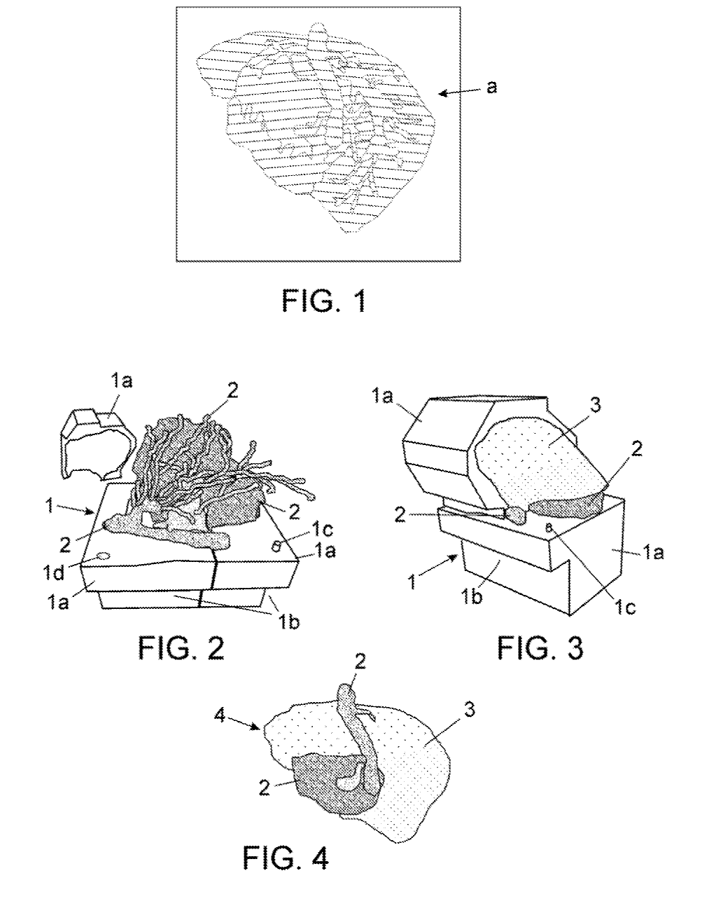

Next, in a second stage, specialized software has been developed for selecting (segmenting) automatically different elements of the organ; in particular, the following elements in the images obtained: the liver parenchyma, hepatic-biliary vasculature differentiating each of the elements and tumor (when appropriate). It will be apparent that other programs known to those skilled in the art may also be used, such as 3D-Doctor or 3D-Slicer software. Using these programs, a three dimensional computerized model of the entire organ is obtained, in this case the liver (a), as seen in FIG. 1.

Subsequently, in a third stage a negative mold (1) of the liver, i.e., the organ assembly, including the hepatic-biliary vasculature and tumors, is designed, with a computer-aided design (CAD) program, such as FreeCAD or Blender, using tools available in these programs.

Preferably, the mold (1) is designed in several sections (1a), as shown in FIG. 2, to further facilitate its removal. These sections (1a) are provided with lugs (1c) and complementary holes (d) for mutual coupling. In addition, the design of the shape of the mold (1) with external shells (1b) optimizes the amount of material used.

A 3D printer, such as the Pruse i3, is then used to print the designed negative mold (1). The material used for printing can be any polymeric material of those commonly used for the manufacture of prototypes obtained from three-dimensional printing machines. In this embodiment, the material used is, for example, PLA (polylactic acid).

Rigid pieces (2) of the internal elements of the model to be provided are then printed in 3D, in the case of the liver, hepatic-biliary vasculature and tumors. The material used can be any rigid material such as the PLA used above or softer materials having up to shore 27A such as TANGO. The printer used for this purpose is a function of the material used. Those rigid pieces are then placed (2) the hepatic-biliary vasculature and tumor in the negative mold (1), designed for such purpose as shown in FIG. 2.

The intrahepatic tumors, i.e., those internal elements that are immersed in the organ, in this case the hepatic parenchyma and that cannot be glued into the mold (1) are placed in position by the use of rods (not shown) or by 3D printing of filaments in the hepatic-biliary vasculature connecting the tumor. And, prior to insertion of the soft material (3) Injected into a subsequent phase, these supports or filaments are removed, the tumors being embedded in the soft material.

Subsequently, a treatment of a demolding facilitator material such as petrolatum is applied on the inside surfaces of the mold and glues on the exposed surfaces of the rigid pieces, in this case of the hepatic-biliary vasculature and tumors, to enhance the adhesion of these elements to the gel later injected. The negative mold (1) is then closed and sealed with insulating material.

It is then injected into the mold (1) with a soft material (3), preferably transparent, such as silicone or silicone gel to create the external element of the organ, in this case the hepatic parenchyma. The volume of injection is a function of the volume of parenchyma of the three-dimensional computer model.

Finally, it is demolded, the piece or anatomical model (4) is formed, as shown in FIG. 3.

Optionally, if demolding is not feasible due to the morphology of the liver or organ being treated, the negative mold is printed (1) in soluble material such as ABS (acrylonitrile butadlene styrene). In such a case, instead of demolding, the mold (1) s submerged into the solvent of the material used to form the piece.

In any event, the anatomical model (4) obtained is an organ, in particular a liver, comprising internal elements, conforming to the hepatic-biliary vasculature and tumors, consisting of rigid pieces (2) of PLA, and external elements, namely the hepatic parenchyma, of transparent soft (3) material, silicone gel or silicone.

### Example (B) for Anatomical Breast Models.

The method of making the anatomical models of breast organs according to the invention comprises, in the first stage, obtaining information about the patient's breast by image diagnostic techniques such as a TAC (Computerized Axial Tomography), Mammography, NMR (Nuclear Magnetic Resonance). Echography or the like.

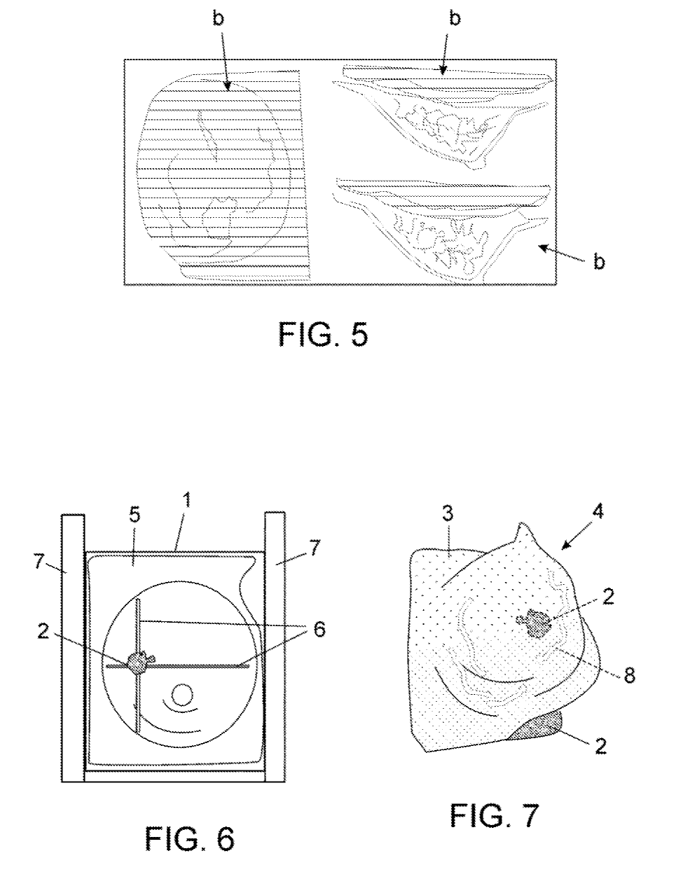

Next, in a second step, by means of specialized software developed to the effect, which selects (segments) automatically the different mammary elements of the image obtained: skin, breast adipose tissue, mammary fibro-glandular tissue, muscle tissue, tumor (if there is one), vasculature and mammary innervation. The segmented elements are a function of the image diagnostic technique used. It will be apparent that other programs known to those skilled in the art may also be used, such as 3D-Doctor or 3D Slicer software. In this way, a three-dimensional computerized model of the entire mammary region is obtained. FIG. 5 shows a schematic representation of different views of the images of this three-dimensional models of the breast (b) being obtained.

Subsequently, in a third stage, a negative mold (1) of the organ, in this case of the breast, is designed, with a computer-aided design (CAD) program such as FreeCAD or Blender, by using tools available in these programs. A 3D printer is then used, such as Prusa i3 for printing the designed negative mold (1). The material used for printing can be any polymeric material of those commonly used for the manufacture of prototypes obtained from three-dimensional printing machines. In this embodiment, the material used is, for example, PLA (polylactic acid).

A coating material (5) is then applied to the surface of the mold, such as latex, to create the skin on the mold (1) as shown in FIG. 6. The volume of material used is a function of the thickness of the skin in the three-dimensional model. Furthermore, prior to the application of such coating (5), various layers of demolding facilitator material such as petrolatum is applied over the surface of the mold (1).

Next, if the presence of fibro-glandular tissue, vasculature and innervation in the final anatomical model is not necessary, the internal element is printed in 3D, in this case the tumor as a rigid piece (2) of the model to be made, and it is spatially placed inside the mold (1) by the use of rods (6) which will be removed prior to the cross-linking of the soft material (3) injected in a subsequent phase. The printing material used can be any rigid material such as the PLA used above or softer materials having up to shore 27A such as those similar to the rubber of the TANGO family. The printer used for this purpose is a function of the material used.

Supports (7) are then placed on the outside of the mold (1), as shown in FIG. 6, for the purpose that, after the subsequent injection of soft material (3) which emulates the adipose tissue, it can be retained above the mold by emulating the lower mammary adipose tissue. The supports (7) may also be printed with the negative breast mold. Then soft material (3) is injected into the open part of the mold such as silicone gels or silicones. The volume of injection is a function of the volume of adipose tissue and muscle tissue of the three-dimensional computer model. The muscle tissue is then printed in 3D, also as an internal element of the model, made with rigid pieces (2), and is spatially placed on the soft material (3), yet not cross-linked. The printing material used can be any rigid material such as the PLA used above or softer materials having up to shore 27A such as TANGO. The printer used for this purpose is a function of the material used. Finally, it is demolded, a piece or anatomical model (4) being formed.

As in the above example, optionally, if demolding is not feasible given the morphology of the breast, the mold (1) is printed in soluble material such as ABS (acrylonitrile butadiene styrene), in which case, instead of demolding, the mold is Immersed into the solvent of the material used to form the piece.

If the presence of additional internal elements (8) is necessary in the final anatomical model, for example, the fibro-glandular tissue, vasculature and innervation, these elements are printed in 3D with soluble material such as ABS that is water soluble and are placed in the mold using adhesives for such purpose. The manufacturing process is then continued by injecting the soft material (3) such as silicone or silicone gel. Once the soft material is cross-linked, the solvent of the soluble material s injected with a fine needle into the zones where these elements are placed, the material being dissolved and leaving the corresponding voids which are filled with silicones or silicone gels of different colors. These elements can also be printed on any rigid material such as the PLA used before or softer materials having up to shore 27A such as TANGO. They are then placed in the mold and the manufacturing process continues with 3D printing of the tumor.

Thereby, in this example, the anatomical model (4) obtained from an organ is a mammary gland comprising internal elements consisting of a tumor and muscle tissue consisting of one or more rigid pieces (2) of PLA, additional internal elements (8) of colored silicon representing fibro-glandular tissue, vasculature and innervation, breast tissue of soft material (3) of silicone gel or transparent silicone and an outer coating (5) of latex, silicones or polyurethanes representing the skin.

Once the nature of this invention, as well as the manner of putting it into practice, has been sufficiently explained, it is not believed necessary to make more extensive explanation so that any person skilled in the art understands its scope and advantages. Moreover, within its essence, it can be practiced in other embodiments which differ in detail from those indicated by way of example, and which will be covered by the protection that is desired as long as it is not modified, changing the fundamental.

1. Method for manufacturing anatomic models of organs or parts thereof for a specific patient, such as liver or breast, applicable for its use in teaching and medical disciplines, such as the planning and simulation of surgeries, comprising:
obtaining information from the anatomical structure of the organ, by image diagnostic techniques, such as CAT (Computerized Axial Tomography) with or without vascular reconstruction, NMR (Nuclear Magnetic Resonance), Echography;
use of medical image processing software for selecting (segmenting), the different elements of the anatomical structure of interest from the images obtained and obtaining a three-dimensional computerized model of the anatomical structure, which is imported into a valid format for 3D printing;
and manufacturing the anatomical model by 3D printing, it is characterized in that this manufacturing by 3D printing comprises the following steps:
design, with a computer-aided design (CAD) program, of a negative mold of the organ assembly;
printing, with a 3D printer, of the designed negative mold;
printing, with a 3D printer of rigid parts corresponding to internal elements of the model provided;
placement of those rigid pieces in the negative mold;
application of a treatment of demolding facilitator material such as petrolatum on the internal surfaces of the mold and glues on the exposed surfaces of the rigid pieces;
the negative mold is closed and sealed with insulating material;
soft material is injected into the mold;
it is demolded, the anatomical piece or model being formed.

2. Method for manufacturing anatomical models, according to claim 1, characterized in that the soft material being injected is silicone or silicone gel.

3. Method for manufacturing anatomical models, according to claim 1, characterized in that the mold is designed in several sections which later facilitate demolding.

4. Method for manufacturing anatomical models, according to claim 1, characterized in that the shape of the mold presents external shells which optimize the amount of material used.

5. Method for manufacturing anatomical models, according to claim 1, characterized in that the material used for the printing of the mold is PLA (polylactic acid).

6. Method for manufacturing anatomical models, according to claim 1, characterized in that the material in which the negative mold is printed is soluble and, for demolding, the mold is submerged into the solvent of the material used.

7. Method for manufacturing anatomical models, according to claim 6, characterized in that the material in which the negative mold is printed is ABS (acrylonitrile butadiene styrene).

8. Method for manufacturing anatomical models, according to claim 1, characterized in that the material used for 3D printing of the rigid parts of the internal elements of the model is PLA.

9. Method for manufacturing anatomical models, according to claim 1, characterized in that the soft material is transparent.

10. Method for manufacturing anatomical models, according to claim 1, characterized in that those internal elements formed by rigid pieces which are immersed in the organ and cannot be glued into the mold are placed in position by the use of rods, and, prior to insertion of the soft material injected in a subsequent phase, they are removed, remaining embedded in the soft material.

11. Method for manufacturing anatomical models, according to claim 1, characterized in that those internal elements formed by rigid pieces which are immersed in the organ and cannot be glued into the mold are placed in position by 3D printing of filaments and, prior to insertion of the soft material injected in a subsequent phase, they are removed and embedded in the soft material.

12. Method for manufacturing anatomical models, according to claim 1, characterized in that, when the organ has skin, such as a mammary gland, prior to placing the rigid pieces, a coating material is applied over the surface of the mold and prior to the application of this coating several layers of demolding facilitator material, such as petrolatum are applied over the surface of the mold.

13. Method for manufacturing anatomical models, according to claim 12, characterized in that the coating material is latex, silicones or polyurethanes.

14. Method for manufacturing anatomical models, according to claim 1, characterized in that, when the organ is a mammary gland, supports are placed on the outside of the mold, prior to injection of the soft material, so that, after the subsequent injection of soft material, it emulates the adipose tissue.

15. Method for manufacturing anatomical models, according to claim 14, characterized in that the supports are printed with the negative mold of the breast.

16. Method for manufacturing anatomical models, according to claim 1, characterized in that when the presence of additional internal elements is necessary in the final anatomical model, these elements are printed in 3D with water soluble material and placed in the mold using adhesives; the manufacturing process is then continued by injecting the soft material and, once cross-linked, the solvent of the soluble material is injected with a fine needle into the zones where these elements are placed leaving the corresponding voids that are filled with silicones or silicon gels of different colors.

17. Anatomical model, obtained by a method as described in claim 1, characterized in that it is a liver comprising internal elements, making up the hepatic-biliary vasculature and tumors, comprising rigid pieces, and external elements, namely the hepatic parenchyma, of soft material.

18. Anatomical model, obtained by a method as described in claim 1, characterized in that it is a mammary gland comprising internal elements consisting of a tumor and muscle tissue, comprising one or more rigid pieces, breast tissue of soft material and an outer coating which represents the skin.

19. Anatomical model, according to claim 18, characterized in that it comprises additional internal elements which represent fibro-glandular tissue, vasculature and innervation.
